(19) United States
(12) Patent Application Publication
(10) Pub. No.: US 2019/0008930 A1
(43) Pub. Date: Jan. 10, 2019

(54) MYCOBACTERIUM LYSTERASE: A NOVEL TREATMENT FOR ACNE

(71) Applicants: The Regents of the University of California, Oakland, CA (US); University of Pittsburgh - Of The Commonwealth System of Higher Education, Pittsburgh, PA (US)

(72) Inventors: Robert L. Modlin, Sherman Oaks, CA (US); Graham F. Hatfull, Pittsburgh, PA (US); Dominique J. Bardeau, Pittsburgh, PA (US); Anil K. Ojha, Pittsburgh, PA (US)

(21) Appl. No.: 15/748,762

(22) PCT Filed: Jul. 28, 2016
(86) PCT No.: PCT/US2016/044427

§ 371 Date: Jan. 30, 2018

Related U.S. Application Data
(60) Provisional application No. 62/199,674, filed on Jul. 31, 2015.

(51) Int. Cl. A61K 38/47; A61K 9/00; A61K 47/10; A61K 47/02; A61P 17/10

(52) U.S. Cl. CPC A61K 38/47; A61K 9/0014; A61P 17/10; A61K 47/10; A61K 47/02; C12Y 302/01017

(57) ABSTRACT

Certain aspects of the invention relate to methods for preventing or treating a skin condition in a subject, comprising administering a composition comprising a lysterase to the subject. Some aspects of the invention relate to pharmaceutical compositions comprising a lysterase. Some aspects of the invention relate to methods for producing a pharmaceutical composition, comprising expressing a lysterase in a recombinant cell and purifying the lysterase.

### RELATED APPLICATIONS

This application claims the benefit of U.S. provisional application 62/199,674, filed on Jul. 31, 2015, which is hereby incorporated by reference in its entirety.

### GOVERNMENT SUPPORT

This invention was made with Government support under AR060382, awarded by the National Institutes of Health. The Government has certain rights in the invention.

### BACKGROUND

Acne vulgaris is a serious skin disease that afflicts about 45 million people in the United States, and more than 80% of people in the U.S. report suffering from acne at some point in their lives. U.S. consumers spend more than $1.2 billion each year on acne treatment. Although not typically life threatening, acne has a strong impact of patient's self-esteem and can have longer term effects such as scarring.

Propionibacterium acnes is strongly implicated as a causal component of acne disease. P. acnes is present in about 100-fold higher concentrations in acne patients as compared to aged matched counterparts, and antibiotic treatment often provides relief. However, antibiotic therapeutics are becoming less effective with the increased prevalence of antibiotic resistant strains. There is clearly a need for new therapeutic approaches for the control of acne.

### SUMMARY

In some aspects, the invention relates to a method for preventing or treating a skin condition in a subject, comprising administering a composition comprising a lysterase to the subject. The skin condition may be acne.

In some aspects, the invention relates to a pharmaceutical composition comprising a lysterase.

In some aspects, the invention relates to a method for making a pharmaceutical composition, comprising expressing a lysterase in a recombinant cell; and purifying the lysterase, i.e., wherein the pharmaceutical composition comprises the lysterase.

### BRIEF DESCRIPTION OF THE DRAWINGS

FIG. 1. Bxb1 or D29 lysterases were added to a culture of M. smegmatis (top panel) and a culture of M. tuberculosis (bottom panel) and incubated for the indicated times (minutes). Cultures were then diluted and spots of each dilution were plated for viable cell growth. The Bxb1 or D29 lysterases killed both cultures of bacteria.

FIG. 2. Bxb1 or D29 lysterases were added to a culture of E. coli and incubated for the indicated times (minutes). Cultures were then diluted and spots of each dilution were plated for viable cell growth. No killing of E. coli was observed.

FIG. 3. Bxb1 or D29 lysterases were added to a culture of Serratia marcescens and incubated for the indicated times (minutes). Cultures were then diluted and spots of each dilution were plated for viable cell growth. No killing of S. marcescens was observed.

FIG. 4. Bxb1 or D29 lysterases were added to a culture of R. erythropolis, and incubated for the indicated times (minutes). Cultures were then diluted and spots of each dilution were plated for viable cell growth. Bxb1 lysterase killed R. erythropolis, although it required high protein concentrations and long incubation times.

FIG. 5. Bxb1 or D29 lysterases was added to a culture of P. acnes and incubated for the indicated times (minutes). Cultures were then diluted and spots of each dilution were plated for viable cell growth. Bxb1 lysterase killed P. acnes within 5 mins of incubation at 2 μM.

FIG. 6. Bxb1 or D29 lysterases were added to a culture of Staphylococcus epidermidis and incubated for the indicated times (minutes). Cultures were then diluted and spots of each dilution were plated for viable cell growth. No killing of S. epidermidis was observed.

FIG. 7. Bxb1 or D29 lysterases was added to a culture of Corynebacterium vitaeruminis and incubated for the indicated times (minutes). Cultures were then diluted and spots of each dilution were plated for viable cell growth. Both Bxb1 and D29 lysterase killed C. vitaeruminis efficiently.

FIG. 8. Bxb1 or D29 lysterases was added to a culture of Corynebacterium glutamicum and incubated for the indicated times (minutes). Cultures were then diluted and spots of each dilution were plated for viable cell growth. Both Bxb1 and D29 lysterase killed C. glutamicum efficiently, although the Bxb1 lysterase appeared to be more active against C. glutamicum than the D29 lysterase.

### DETAILED DESCRIPTION

Bacteriophages are the most numerous biological entities in the biosphere, with rich genetic diversity. Several bacteriophages of P. acnes were isolated and characterized as disclosed herein P. acnes bacteriophages share several genes with the mycobacteriophages, the viruses of mycobacterial hosts, reflecting the phylogenetic relationships of their hosts; both Mycobacterium and Propionibacterium are classified within the Order Actinomycetales. Although the mycobacteria are members of the Gram-positive Actinomycetales, they are unusual in possessing a mycolic acid-rich outer membrane that is covalently attached to the inner arabinogalactan-peptidoglycan complex through a mycolylarabinogalactan bond. Most mycobacteriophages encode two enzymes for lysis of the host's membrane, an endolysin (lysin A) and a lysterase (lysin B or LysB), P. acnes phages encode only an endolysin (Payne et al., 2009, Payne & Hatfull, 2012 and Hatfull, 2012). The simplest interpretation of this structural difference is that mycobacteriophage-encoded lysterases are responsible for digestion of mycolic acid-containing cell wall components that are unique to the mycobacteria, and are not found in other bacteria such as P. acnes. Specifically, the mycolylarabinogalactan bond may be the lysterase substrate. This bond is only present in bacteria within the phylum Actinobacteria, and in the mycobacteria, and is known to be essential for growth—in fact, its synthesis is the target of the first line antituberculosis drug, isoniazid.

Comparative analysis of mycobacteriophage genomes reveals them to be highly diverse. Previously, 6,858 predicted protein products were assorted into 1,523 families (Phams) of related sequences and their distributions among the mycobacteriophages analyzed. Representative examples of mycobacteriophages include L5 (Hatfull and Sarkis, 1993), D29 (Ford et al., 1998), Ms6 Lys B (Gil et al., 2008), and TM4 (Ford et al., 1998). The lysA family (Pham 66), and the LysA proteins are predicted from sequence comparisons to have peptidoglycan hydrolyzing activity (Pedulla et al. 2003). The lysB gene, which encodes lysin B (Lys B), is located downstream of lysA, is also implicated in lysis, primarily because of its linkage to lysA and the demonstration of lipolytic activity by Ms6 Lys B (Gil et al., 2008). LysB homologs were found in 56 of 60 completely sequenced mycobacteriophage genomes and are located downstream of lysA. Some of the intervening genes encode putative holins and exhibit holin-like function (e.g. D29 gene 11), whereas others (e.g. Omega gene 51) code for putative homing endonuclease HNH motifs.

Lysterases useful in the compositions and methods described herein include a mycolylarabinogalactan esterase, the lysterase encoded by phage D29 that cleaves the linkage of the mycolic acid outer membrane to arabinogalactan of mycobacteria, releasing free mycolic acids and thereby promoting lysis (Payne et al., 2009). Another suitable lysterase is a lysterase encoded by the mycobacteriophage Bxb1, which cleaves the linkage of the mycolic acid outer membrane to arabinogalactan of mycobacteria.

Other mycobacteria contain mycolic acids, and they are collectively referred to as the mycolata; members include Rhodococcus spp., Gordonia spp., Nocardia spp., and Corynbacterium spp. A robust correlation exists between the presence of the lysterase gene in a phage genome and essential cell wall mycolic acids in the host.

Some aspects of the invention relate to the unexpected finding that lysterases also potently kill of P. acnes.

### Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “preventing” is art-recognized, and when used in relation to a condition such as a local recurrence (e.g., blemish), a disease such as acne, or an injury such as scarring, is well understood in the art, and includes administration of a composition which reduces the frequency of, or delays the onset of, symptoms of a medical condition in a subject relative to a subject which does not receive the composition. Thus, prevention of acne includes, for example, reducing the number of detectable blemishes (e.g., lesions) in a population of patients receiving a prophylactic treatment relative to an untreated control population, and/or delaying the appearance of detectable blemishes in a treated population versus an untreated control population, e.g., by a statistically and/or clinically significant amount.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The term “subject” refers to a mammal, including, but not limited to, a human or non-human mammal, such as a bovine, equine, ovine, porcine, canine, lagomorph, feline, or rodent. In certain preferred embodiments, the subject is a human.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

### I. Methods for Preventing or Treating a Skin Condition

In some aspects, the invention relates to a method for preventing or treating a skin condition in a subject, comprising administering a composition comprising a lysterase to the subject. The composition may be any composition described herein. Administering the composition may comprise topically administering the composition, e.g., to the skin.

In some embodiments, the skin condition is acne. The skin condition may be scarring, e.g., scarring associated with acne.

In some embodiments, the condition is associated with a bacterium. The bacterium may be Actinomycetales. The bacterium may comprise mycolic acid, or the bacterium may be substantially free of mycolic acid. In some embodiments, the bacterium is Propionibacterium, such as Propionibacterium acnes.

In some embodiments, the skin condition is associated with a bacterium and the bacterium is not Propionibacterium acnes. In some embodiments, the skin condition is associated with a bacterium and the bacterium is not Propionibacterium. In some embodiments, the skin condition is associated with a bacterium and the bacterium is not Actinomycetales.

### II. Pharmaceutical Compositions Comprising a Lysterase

In some aspects, the invention relates to a pharmaceutical composition comprising a lysterase, e.g., for use in treating a skin condition.

In some embodiments, the lysterase is lysin B. The lysterase may be D29 lysterase or Bxb1 lysterase.

The lysterase may be the lysterase of a bacteriophage. The lysterase may be the lysterase of a mycobacteriophage. In some embodiments, the bacteriophage cannot infect any strain of Propionibacterium acnes, and yet the lysterase is active against at least one strain of Propionibacterium acnes. In some embodiments, the bacteriophage cannot infect a strain of Propionibacterium acnes, and yet the lysterase is active against that strain. The lysterase may be a lysterase encoded by the D29 phage or the Bxb1 phage. The lysterase may be the lysterase of a bacteriophage that can infect a species of Propionibacterium. In some embodiments, the lysterase is the lysterase of a bacteriophage that can infect a strain of Propionibacterium acnes. In some embodiments, the bacteriophage can infect a strain of Propionibacterium acnes, and the lysterase is active against that strain. The composition may comprise a bacteriophage. In preferred embodiments, the composition does not comprise a bacteriophage.

The composition may comprise about 1 nM lysterase to about 1 mM lysterase, such as about 10 nM lysterase to about 100 μM lysterase, or about 100 nM lysterase to about 10 μM lysterase. The composition may comprise about 100 nM lysterase, about 200 nM lysterase, about 300 nM lysterase, about 400 nM lysterase, about 500 nM lysterase, about 600 nM lysterase, about 700 nM lysterase, about 800 nM lysterase, about 900 nM lysterase, about 1.0 μM, about 1.5 μM lysterase, about 2.0 μM lysterase, about 2.5 μM lysterase, about 3.0 μM lysterase, about 3.5 μM lysterase, about 4.0 μM lysterase, about 4.5 μM lysterase, about 5.0 μM lysterase, about 5.5 μM lysterase, about 6.0 μM lysterase, about 6.5 μM lysterase, about 7.0 μM lysterase, about 7.5 μM lysterase, about 8.0 μM about 8.5 μM lysterase, lysterase, about 9.0 μM lysterase, about 9.5 μM lysterase, or about 10 μM lysterase.

In some aspects, the invention relates to a pharmaceutical composition comprising a lysterase. The pharmaceutical composition may be formulated for topical administration. The formulation may be a liquid, gel, or cream.

Exemplary identities of various constituents of the topical formulations of some embodiments of the present invention are described below.

### 1. Vehicles, Solvents, and Diluents

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In one embodiment, formulations without methanol, ethanol, propanols, or butanols are desirable.

### 2. Surfactants and Emulsifiers

Many topical formulations contain chemical emulsions which use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-10 phosphate, methylbenzethonium chloride, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), ceteth-20, Brij S10 (polyethylene glycol octadecyl ether, average Mₙ˜711), and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101 and b is about 56)). Appropriate combinations or mixtures of such surfactants may also be used according to the present invention.

Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (cetearyl alcohol), ceteareth-12, ceteareth-20, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-75 stearate, PEG-100 stearate, and PEG-100 stearate, ceramide 2, ceramide 3, stearic acid, cholesterol, steareth-2, and steareth-20, or combinations/mixtures thereof, as well as cationic emulsifiers like stearamidopropyl dimethylamine and behentrimonium methosulfate, or combinations/mixtures thereof.

### 3. Moisturizers, Emollients, and Humectants

One of the most important aspects of topical products in general, and cosmetic products in particular, is the consumer's perception of the aesthetic qualities of a product. For example, while white petrolatum is an excellent moisturizer and skin protectant, it is rarely used alone, especially on the face, because it is greasy, sticky, does not rub easily into the skin and may soil clothing. Consumers highly value products which are aesthetically elegant and have an acceptable tactile feel and performance on their skin.

Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800.

Suitable emollients or humectants for use in the formulations of the present invention include, but are not limited to, panthenol, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, and 1,3-bis(N-2-(hydroxyethyl)palmitoylamino)-2-hydroxypropane.

In addition, appropriate combinations and mixtures of any of these moisturizing agents and emollients may be used in accordance with the present invention.

### 4. Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols, tocopheryl acetate, sodium ascorbate/ascorbic acid, ascorbyl palmitate, propyl gallate, and chelating agents like EDTA (e.g., disodium EDTA), citric acid, and sodium citrate.

In certain embodiments, the antioxidant or preservative comprises (3-(4chlorophenoxy)-2-hydroxypropyl)carbamate.

In certain embodiments, antioxidants or preservatives of the present invention may also function as a moisturizer or emollient, for example.

In addition, combinations or mixtures of these preservatives or anti-oxidants may also be used in the formulations of the present invention.

### 5. Combination Agents

The composition can also contain any other agent that has a desired effect when applied topically to a mammal, particularly a human. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones. Mixtures of any of these active agents may also be employed. Additionally, dermatologically-acceptable salts and esters of any of these agents may be employed.

It is known that antimicrobials such as antibiotics can be bactericidal or bacteriostatic, limiting and reducing the quantity of bacteria in our bodies. Retinoids also reduce the amount of bacteria in pilosebaceous units and microcomedones. Light based therapies work via photothermal heating, photochemical inactivation of bacteria, and various photoimmunological reactions to improve clinical skin outcomes. In general, the above therapies directly act to reduce the amount of pathogenic bacteria in a patient. Thus, the invention proposes that any such therapy that achieves the same goal of reducing the number of pathogenic organisms, when used in combination with subsequent topical probiotic treatment, would lead to replacement of the pathogenic microflora involved in the diseased state with natural microflora enriched in healthy skin or mucous membranes, or less pathogenic species occupying the same ecological niche as the type causing a disease state.

Suitable antibacterial compounds include capreomycins, including capreomycin IA, capreomycin IB, capreomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefmenoxime, cefmetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cephalosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dirithromycin, doxycyclin, epicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A1, lincomycin, lomefloxacin, loracarbef, lymecycline, meropenam, metampicillin, methacycline, methicillin, mezlocillin, micronomicin, midecamycins such as midecamycin A1, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, and protected forms thereof.

Suitable anti-fungal compounds include ketoconazole, miconazole, fluconazole, clotrimazole, undecylenic acid, sertaconazole, terbinafine, butenafine, clioquinol, haloprogin, nystatin, naftifine, tolnaftate, ciclopirox, amphotericin B, or tea tree oil and analogs, derivatives, pharmaceutically acceptable salts, esters, prodrugs, and protected forms thereof.

Suitable antiviral agents include acyclovir, azidouridine, anismoycin, amantadine, bromovinyldeoxusidine, chlorovinyldeoxusidine, cytarabine, delavirdine, didanosine, deoxynojirimycin, dideoxycytidine, dideoxyinosine, dideoxynucleoside, desciclovir, deoxyacyclovir, efavirenz, enviroxime, fiacitabine, foscamet, fialuridine, fluorothymidine, floxuridine, ganciclovir, hypericin, idoxuridine, interferon, interleukin, isethionate, nevirapine, pentamidine, ribavirin, rimantadine, stavudine, sargramostin, suramin, trichosanthin, tribromothymidine, trichlorothymidine, trifluorothymidine, trisodium phosphomonoformate, vidarabine, zidoviridine, zalcitabine and 3-azido-3-deoxythymidine and analogs, derivatives, pharmaceutically acceptable salts, esters, prodrugs, and protected forms thereof.

Other suitable antiviral agents include 2′,3′-dideoxyadenosine (ddA), 2′,3′-dideoxyguanosine (ddG), 2′,3′-dideoxycytidine (ddC), 2′,3′-dideoxythymidine (ddT), 2′3′-dideoxy-dideoxythymidine (d4T), 2′-deoxy-3′-thia-cytosine (3TC or lamivudime), 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, 2′3′-dideoxy-2′,3′-didehydro-2′fluorothymidine (Fd4T), 2′3′-dideoxy-2′-beta-fluoroadenosine (F-ddA), 2′3′-dideoxy-2′-beta-fluoro-inosine (F-ddI), and 2′,3′-dideoxy-2′-beta-flurocytosine (F-ddC). In some embodiments, the antiviral agent is selected from trisodium phosphomonoformate, ganciclovir, trifluorothymidine, acyclovir, 3′-azido-3′-thymidine (AZT), dideoxyinosine (ddI), and idoxuridine and analogs, derivatives, pharmaceutically acceptable salts, esters, prodrugs, and protected forms thereof.

### 6. Buffer Salts

Suitable buffer salts are well-known in the art. Examples of suitable buffer salts include, but are not limited to sodium citrate, citric acid, sodium phosphate monobasic, sodium phosphate dibasic, sodium phosphate tribasic, potassium phosphate monobasic, potassium phosphate dibasic, and potassium phosphate tribasic.

### 7. Viscosity Modifiers

Suitable viscosity adjusting agents (i.e., thickening and thinning agents or viscosity modifying agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these viscosity adjusters may be utilized according to the present invention.

### 8. Additional Constituents

Additional constituents suitable for incorporation into the emulsions of the present invention include, but are not limited to: skin protectants, adsorbents, demulcents, emollients, moisturizers, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate).

For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of peptides that interact with protein structures of the dermal-epidermal junction include palmitoyl dipeptide-5 diaminobutyloyl hydroxythreonine and palmitoyl dipeptide-6 diaminohydroxybutyrate.

Examples of skin soothing agents include, but are not limited to algae extract, mugwort extract, stearyl glycyrrhetinate, bisabolol, allantoin, aloe, avocado oil, green tea extract, hops extract, chamomile extract, colloidal oatmeal, calamine, cucumber extract, and combinations thereof.

In certain embodiments, the compositions comprise bergamot or bergamot oil. Bergamot oil is a natural skin toner and detoxifier. In certain embodiments, it may prevent premature aging of skin and may have excellent effects on oily skin conditions and acne.

Examples of vitamins include, but are not limited to, vitamins A, D, E, K, and combinations thereof. Vitamin analogues are also contemplated; for example, the vitamin D analogues calcipotriene or calcipotriol.

In certain embodiments, the vitamin may be present as tetrahexyldecyl ascorbate. This compound exhibits anti-oxidant activity, inhibiting lipid peroxidation. In certain embodiments, use can mitigate the damaging effects of UV exposure. Studies have shown it to stimulate collagen production as well as clarifying and brightening the skin by inhibiting melanogenesis (the production of pigment) thereby promoting a more even skin tone.

Examples of sunscreens include, but are not limited to, p-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, 4methylbenzylidene camphor, methylene bis-benzotriazolyl tetramethylbutylphenol, bisethylhexyloxyphenol methoxyphenyl triazine, terephthalylidene dicamphor sulfonic acid, drometrizole trisiloxane, disodium phenyl dibenzimidazole tetrasulfonate, diethylamino hydroxybenzoyl hexyl benzoate, octyl triazone, diethylhexyl butamido triazone, polysilicone-15, and combinations thereof.

Suitable fragrances and colors may be used in the formulations of the present invention. Examples of fragrances and colors suitable for use in topical products are known in the art.

Suitable immunomodulators include, but are not limited to, tetrachlorodecaoxide, deoxycholic acid, tacrolimus, pimecrolimus, and beta-glucan.

In certain embodiments, palmitoyl-lysyl-valyl-lysine bistrifluoroacetate is added. This peptide stimulates collagen synthesis in human fibroblasts.

Often, one constituent of a composition may accomplish several functions. In one embodiment, the present invention relates to constituents that may act as a lubricant, an emollient, or a skin-penetrating agent. In one embodiment, the multi-functional constituent is socetyl stearate, isopropyl isostearate, isopropyl palmitate, or isopropyl myristate.

### III. Methods for Making a Pharmaceutical Composition Comprising a Lysterase

In some aspects, the invention relates to a method for making a pharmaceutical composition comprising a lysterase, comprising expressing a lysterase in a recombinant cell and purifying the lysterase. The cell may be, for example, E. coli.

### EXEMPLIFICATION

### Example 1

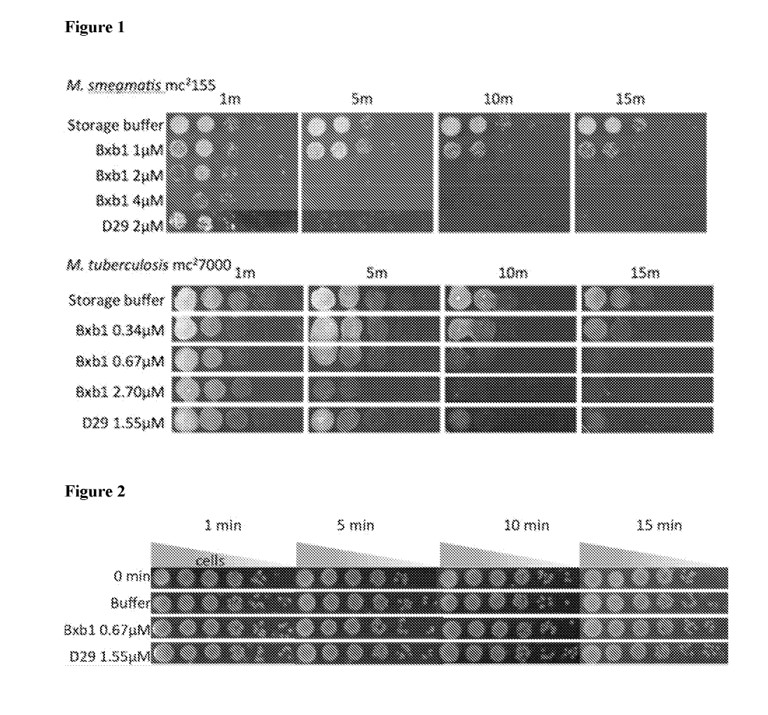

The Bxb1 and D29 lysterases were cloned and overexpressed in E. coli to near homogeneity. Addition of either lysterase to a culture of M. smegmatis resulted in substantial killing, and similar observations were made using M. tuberculosis (FIG. 1). Importantly, controls using either boiled protein or similar preparations of a catalytically defective mutant displayed no killing.

To test specificity of lysterase action, the addition of exogenous lysterase to both E. coli and Serratia marcescens was determined. E. coli is a gram-negative and far-distant relative of the mycobacteria, and S. marcescens is also a gram-negative bacterium. No killing was observed for either the D29 or Bxb1 lysterase in either strain (FIGS. 2 and 3).

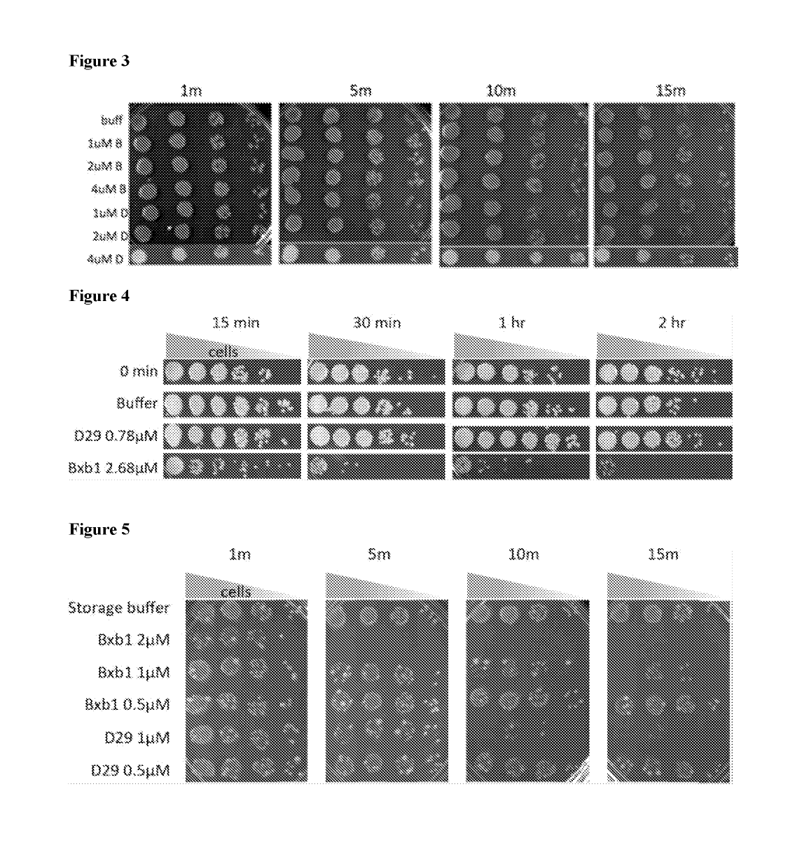

The ability of the lysterases to kill other strains within the Order Actinomycetales was then determined. Actinomycetales includes both bacteria classified as the Mycolata (the myolic acid containing strains) and those that do not contain mycolic acid. First, the lysterase killing of Rhodococcus globerulus, a strain that contains mycolic acids, was tested (FIG. 4). Little or no killing of R. globerulus by D29 lysterase was observed, but efficient killing with the Bxb1 lysterase was observed (FIG. 4). This result is surprising given the general differences between the mycolic acid containing components of the mycobacterial and Rhodococcus cell walls.

The ability of the lysterases to kill P. acnes, a clinically relevant strain of the Actinomycetales, was determined. P. acnes does not contain mycolic acids, and thus, there was no expectation of lysterase activity against it. Further, phage of P. acnes also do not contain lysterase genes. Surprisingly, both the D29 and Bxb1 lysterases efficiently killed P. acnes, with 2 μM of Bxb1 lysterase killing most P. acnes cells within 5 minutes of exposure (FIG. 5).

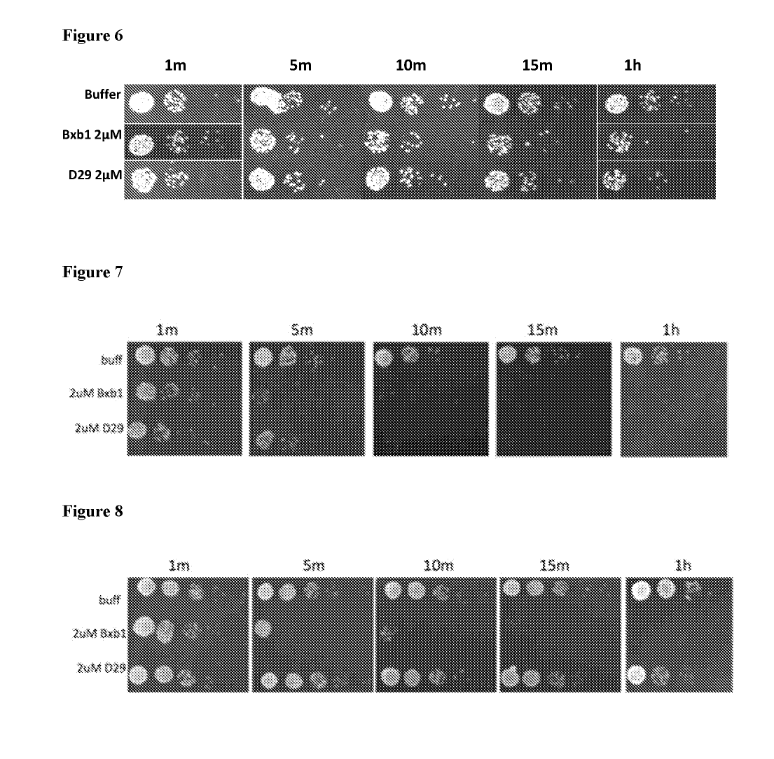

Lysterases were also tested for activity against several other bacterial species. Of note, no activity was observed against Staphylococcus epidermidis (FIG. 6). The only tested strains that displayed sensitivity to the lysterases fell within the order Actinomycetales, including Corynebacterium vitaeruminis and Corynebacterium glutamicum, although the D29 enzyme appeared to be less active against the latter strain (FIGS. 7 and 8).

### INCORPORATION BY REFERENCE

All of the scientific articles, patents, and published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method for preventing or treating a skin condition in a subject, comprising administering a composition comprising a lysterase to the subject.

2. The method of claim 1, wherein administering the composition comprises topically administering the composition.

3. The method of claim 1 or 2, wherein the skin condition is associated with a bacterium.

4. The method of claim 3, wherein the bacterium is Actinomycetales.

5. The method of claim 3 or 4, wherein the bacterium is substantially free of mycolic acid.

6. The method of claim 4 or 5, wherein the bacterium is Propionibacterium acnes.

7. The method of any one of the preceding claims, wherein the skin condition is acne.

8. The method of any one of the preceding claims, wherein the lysterase is lysin B.

9. The method of any one of the preceding claims, wherein the lysterase is D29 lysterase or Bxb1 lysterase.

10. The method of any one of the preceding claims, wherein the composition comprises about 1 nM lysterase to about 1 mM lysterase.

11. The method of claim 10, wherein the composition comprises about 10 nM lysterase to about 100 μM lysterase.

12. The method of claim 11, wherein the composition comprises about 100 nM lysterase to about 10 μM lysterase.

13. The method of any one of the preceding claims, wherein the composition does not comprise a phage.

14. A pharmaceutical composition comprising a lysterase.

15. The pharmaceutical composition of claim 14, wherein the composition is formulated for topical administration.

16. The pharmaceutical composition of claim 14 or 15, wherein the lysterase is lysin B.

17. The pharmaceutical composition of any one of claims 14 to 16, wherein the lysterase is D29 lysterase or Bxb1 lysterase.

18. The pharmaceutical composition of any one of claims 14 to 17, wherein the composition comprises about 1 nM to about 1 mM lysterase.

19. The pharmaceutical composition of claim 18, wherein the composition comprises about 10 nM to about 100 μM lysterase.

20. The pharmaceutical composition of claim 19, wherein the composition comprises about 100 nM to about 10 μM lysterase.

21. The pharmaceutical composition of any one of claims 14 to 20, further comprising water from about 10% to about 95% by weight.

22. The pharmaceutical composition of any one of claims 14 to 21, further comprising glycerin from about 1% to about 20% by weight.

23. The pharmaceutical composition of any one of claims 14 to 22, further comprising propylene glycol from about 1% to about 40% by weight.

24. The pharmaceutical composition of any one of claims 14 to 23, further comprising phosphate at a concentration from about 0.5 mM to about 500 mM.

25. The pharmaceutical composition of any one of claims 14 to 24, further comprising EDTA at a concentration from about 1 mM to about 100 mM.

26. The pharmaceutical composition of any one of claims 14 to 25, wherein the composition does not comprise a phage.

27. A method for making the pharmaceutical composition of any one of claims 14 to 26, comprising:
expressing the lysterase in a recombinant cell; and
purifying the lysterase.
